(19) United States
(12) Patent Application Publication
(10) Pub. No.: US 2022/0225921 A1
(43) Pub. Date: Jul. 21, 2022

(54) MICROELECTRODE, OCCLUDING DEVICE, MICROELECTRODE SYSTEM, METHOD FOR MANUFACTURING MICROELECTRODE, AND METHOD FOR USING MICROELECTRODE

(71) Applicant: TSINGHUA UNIVERSITY, Beijing (CN)

(72) Inventors: Huaqiang WU, Beijing (CN); Jianshi TANG, Beijing (CN); Jian YUAN, Beijing (CN)

(73) Assignee: TSINGHUA UNIVERSITY, Beijing (CN)

(21) Appl. No.: 17/605,122

(22) PCT Filed: Nov. 19, 2020
(86) PCT No.: PCT/CN2020/130087

§ 371 Date: Oct. 20, 2021

(30) Foreign Application Priority Data
Nov. 29, 2019 (CN) 201911203098.X

(51) Int. Cl. A61B 5/293; A61N 1/05; A61N 1/36

(52) U.S. Cl. CPC A61B 5/293; A61N 1/0529; A61B 2562/168; A61B 2562/164; A61N 1/3605

(57) ABSTRACT

A microelectrode, a method for manufacturing the microelectrode, a method for using the microelectrode, an occluding device, and a microelectrode system are provided. The microelectrode (10) includes a substrate (110) and a conductive layer (120) on the substrate (110), and the conductive layer (120) is configured to conduct an electrical signal. The substrate (110) is a flexible substrate and includes a cavity structure (111), and the cavity structure (111) is configured to contain or release a fluid. The hardness of the substrate (110) in the case where the cavity structure (111) contains the fluid is different from the hardness of the substrate (110) in the case where the cavity structure (111) does not contain the fluid. The microelectrode has good ductility and stable electrical performance, and the microelectrode is easy to be implanted into the biological tissue and not easy to result in the immune reaction of the biological tissue.

This application claims the priority of Chinese Patent Application No. 201911203098.X, filed on Nov. 29, 2019, the entire disclosure of which is incorporated herein by reference as part of the disclosure of this application.

### TECHNICAL FIELD

Embodiments of the present disclosure relate to a microelectrode, an occluding device for the microelectrode, a microelectrode system, a method for manufacturing the microelectrode, and a method for using the microelectrode.

### BACKGROUND

With the development of the nerve microelectrode technology, new achievements have been made in neuroscience and related engineering research, especially in popular research fields such as brain-computer interface and neural prosthesis. In the neural engineering system, the nerve electrode is the key interface between nerve tissue and functional instrument, and the performance of the nerve electrode directly determines the ultimate performance of the whole nerve activity recording system or nerve function reconstruction system. There are two main functions of the nerve electrode, that is, one is to convert nerve activities into electrical signals and record the electrical signals for analysis and research, and the other is to use electrical signals to stimulate or restrain nerve activities for functional electrical stimulation.

### SUMMARY

At least one embodiment of the present disclosure provides a microelectrode, which includes: a substrate, and a conductive layer on the substrate which is configured to conduct an electrical signal. The substrate is a flexible substrate and comprises a cavity structure, the cavity structure is configured to contain or release a fluid, and the hardness of the substrate in the case where the cavity structure contains the fluid is different from the hardness of the substrate in the case where the cavity structure does not contain the fluid.

For example, in the microelectrode provided by at least one embodiment of the present disclosure, the substrate comprises a site region, a transition region, and a connection region. The conductive layer comprises a site part, a conductive part, and a connection part, the site part is configured to collect and/or output the electrical signal, the connection part is configured to input and/or output the electrical signal, and the conductive part is configured to transmit the electrical signal between the site part and the connection part. The site part is located in the site region, the conductive part is located in the transition region, and the connection part is located in the connection region.

For example, in the microelectrode provided by at least one embodiment of the present disclosure, the cavity structure is located in the site region, the transition region, and the connection region.

For example, in the microelectrode provided by at least one embodiment of the present disclosure, one end of the cavity structure is an open end, the other end of the cavity structure is a closed end, the open end is located in the connection region, and the closed end is located in the site region.

For example, in the microelectrode provided by at least one embodiment of the present disclosure, the cavity structure comprises a first cavity and a second cavity, and the first cavity and the second cavity are communicated with each other. The first cavity has a cuboid shape, and is located in the transition region, the connection region, and the site region. The second cavity is located in the site region, and the second cavity has a tip shape at the closed end.

For example, in the microelectrode provided by at least one embodiment of the present disclosure, the tip shape comprises a triangular prism shape, a tapered shape, or an inverted trapezoidal shape.

For example, in the microelectrode provided by at least one embodiment of the present disclosure, the width of the first cavity ranges from 30 microns to 90 microns.

For example, in the microelectrode provided by at least one embodiment of the present disclosure, the height of the first cavity ranges from 10 microns to 90 microns.

For example, in the microelectrode provided by at least one embodiment of the present disclosure, the length of the cavity structure is equal to the length of the substrate.

For example, in the microelectrode provided by at least one embodiment of the present disclosure, the fluid comprises air, single component gas, or liquid.

For example, in the microelectrode provided by at least one embodiment of the present disclosure, the material of the substrate comprises a polymer, and the polymer comprises polyimide, parylene, or photo-sensitive epoxy resin photoresist.

For example, in the microelectrode provided by at least one embodiment of the present disclosure, the substrate comprises an insulating wall, the insulating wall surrounds the cavity structure, and the thickness of the insulating wall ranges from 1 micron to 6 microns.

For example, in the microelectrode provided by at least one embodiment of the present disclosure, the site part comprises a plurality of electrode points, the conductive part comprises a plurality of connection lines, and the connection part comprises a plurality of connection points. The plurality of electrode points, the plurality of connection lines, and the plurality of connection points are in one-to-one correspondence, one end of the connection line is electrically connected to a corresponding electrode point, and the other end of the connection line is electrically connected to a corresponding connection point.

For example, the microelectrode provided by at least one embodiment of the present disclosure further comprises a protective layer. The protective layer covers the conductive part and exposes the site part and the connection part.

At least one embodiment of the present disclosure also provides an occluding device for the microelectrode according to any embodiments above. The occluding device is configured to close the cavity structure after the cavity structure is filled with the fluid so that the cavity structure contains the fluid, and to open the cavity structure so that the fluid in the cavity structure flows out.

At least one embodiment of the present disclosure also provides a microelectrode system, which includes the microelectrode according to any embodiments above and the occluding device according to any embodiments above.

For example, the microelectrode system provided by at least one embodiment of the present disclosure further includes a fluid control device. The fluid control device is configured to inject the fluid into the cavity structure or suck the fluid out of the cavity structure.

At least one embodiment of the present disclosure also provides a method for manufacturing the microelectrode according to any embodiments above. The method includes: providing a silicon wafer; forming a first insulating layer on the silicon wafer; forming a filling part on the first insulating layer, in which a shape and size of the filling part are same as a shape and size of the cavity structure; forming a second insulating layer on the first insulating layer, in which the second insulating layer covers the filling part; forming the conductive layer on the second insulating layer; forming a third insulating layer on the second insulating layer, in which the third insulating layer covers the conductive part of the conductive layer and exposes the site part and the connection part of the conductive layer; dissolving the filling part; and separating the first insulating layer from the silicon wafer so as to form the microelectrode. The substrate comprises the first insulating layer and the second insulating layer.

For example, in the method provided by at least one embodiment of the present disclosure, the materials of the first insulating layer, the second insulating layer, and the third insulating layer are the same polymer material.

For example, in the method provided by at least one embodiment of the present disclosure, the material of the filling part is photoresist.

At least one embodiment of the present disclosure also provides a method for using the microelectrode according to any embodiments above, which includes: filling the fluid into the cavity structure of the microelectrode and closing the cavity structure; implanting the microelectrode into biological tissue; and opening the cavity structure and releasing the fluid in the cavity structure.

### BRIEF DESCRIPTION OF THE DRAWINGS

In order to more clearly illustrate the technical schemes of the embodiments of the present disclosure, the drawings of the embodiments will be briefly described in the following. It is obvious that the described drawings below are only related to some embodiments of the disclosure and are not limitative to the disclosure.

FIG. 1A is a schematic block diagram of the microelectrode provided by at least one embodiment of the present disclosure;

FIG. 1B is a schematic perspective view of the microelectrode provided by at least one embodiment of the present disclosure;

FIG. 1C is a top view of the microelectrode provided by at least one embodiment of the present disclosure;

FIG. 2A is a schematic diagram of the cavity structure included in the microelectrode provided by at least one embodiment of the present disclosure;

FIG. 2B is a top view of the cavity structure included in the microelectrode provided by at least one embodiment of the present disclosure;

FIG. 2C is a top view of other forms of cavity structures included in the microelectrode provided by at least one embodiment of the present disclosure;

FIG. 2D is a schematic diagram of an open end of the cavity structure included in the microelectrode provided by at least one embodiment of the present disclosure;

FIG. 2E is a schematic perspective view of another microelectrode provided by at least one embodiment of the present disclosure;

FIG. 3 is a schematic diagram of an occluding device for the microelectrode provided by at least one embodiment of the present disclosure;

FIG. 4A is a schematic block diagram of the microelectrode system provided by at least one embodiment of the present disclosure;

FIG. 4B is a schematic block diagram of another microelectrode system provided by at least one embodiment of the present disclosure;

FIG. 5 is a flowchart of a method for manufacturing the microelectrode provided by at least one embodiment of the present disclosure;

FIGS. 6A-6H are schematic perspective views of the microelectrode provided by at least one embodiment of the present disclosure in the manufacturing process;

FIGS. 7A-7H are schematic cross-sectional views of the microelectrode provided by at least one embodiment of the present disclosure in the manufacturing process; and

FIG. 8 is a flow chart of a method for using the microelectrodes provided by at least one embodiment of the present disclosure.

### DETAILED DESCRIPTION

In order to make the objects, technical schemes and advantages of the embodiments of the present disclosure more clear, the technical solutions of the embodiments of the present disclosure will be described in a clear and full way in connection with the drawings of the embodiments of the present disclosure. Obviously, the described embodiments are some embodiments of the present disclosure, not all embodiments. Based on the described embodiments of the present disclosure, all other embodiments obtained by those of ordinary skill in the art without the use of inventive faculty are within the scope of the present disclosure.

Unless otherwise defined, all the technical and scientific terms used herein have the same meanings as commonly understood by those of ordinary skill in the art to which the present disclosure belongs. The terms “first,” “second,” etc., which are used in the present disclosure, are not intended to indicate any sequence, amount or importance, but used to distinguish various components. Similarly, the terms, such as “a/an,” “the,” “one,” etc., are not intended to indicate the limitation on amounts, but used to denote the presence of at least one. The terms, such as “comprise/comprising,” “include/including,” etc., are intended to specify that the elements or the objects stated before these terms encompass the elements or the objects and equivalents thereof listed after these terms, but not preclude other elements or objects. The terms, such as “connect/connecting/connected,” “couple/coupling/coupled” etc., are not limited to a physical connection or mechanical connection, but may include an electrical connection/coupling, directly or indirectly. The terms, “on,” “under,” “left,” “right,” etc., are only used to indicate relative position relationship, and when the position of the object which is described is changed, the relative position relationship may be changed accordingly.

In the neural engineering system, for example, the neural microelectrode array can be implanted in the brain of an animal to build the brain-computer interface system, so that the animal can directly control the motion of the mechanical arm through the brain's thoughts. Because the size of the nerve cell body is small, and its diameter is usually between 10 microns and 50 microns, it is difficult to detect nerve activities using conventional macro-electrodes, thus, it is required to process micro-electrodes on micron scale.

At present, in the technical development of the nerve microelectrode, the silicon-based nerve microelectrode has been developed for a long time, and its biocompatibility has been widely recognized. However, the hardness of the silicon-based material is largely higher than that of brain tissue, which causes the immune response of brain tissue after implantation, which is unfavorable for long-term stable recording of electroencephalogram signals. Therefore, the development of flexible electrodes with the same hardness as brain tissue has been paid attention to and made continuous progress. However, the flexible nerve microelectrode is too soft and is easy to be bent, and cannot be directly inserted into brain tissue like the silicon-based nerve microelectrode, thus there is a technical difficulty in implantation.

For the implantation difficulties existing in the use of the flexible electrodes, the embodiments of the present disclosure provide a microelectrode capable of containing and releasing a fluid. After the fluid is filled, the hardness of the microelectrode is at least one order of magnitude higher than the hardness of biological tissue (such as brain tissue). Like the silicon-based nerve microelectrode, the microelectrode can be implanted directly by the precise three-dimensional displacement thruster. After the fluid is released, the hardness of the microelectrode is close to the hardness of brain tissue, so that the microelectrode can coexist with brain tissue naturally, thereby avoiding the rejection of brain tissue and the inflammation caused by the immune reaction, because of excessive hardness.

At least one embodiment of the present disclosure provides a microelectrode, and the microelectrode includes a substrate and a conductive layer. The conductive layer is on the substrate and configured to conduct an electrical signal. The substrate is a flexible substrate and includes the cavity structure which is configured to contain or release a fluid, and the hardness of the substrate in the case where the cavity structure contains the fluid is different from the hardness of the substrate in the case where the cavity structure does not contain the fluid.

At least one embodiment of the present disclosure also provides a method for manufacturing the microelectrode and a method for using the microelectrode, an occluding device, and a microelectrode system.

The microelectrode provided by the embodiments of the present disclosure has good ductility and stable electrical performance, and combines the advantage of the mature implantation method of the silicon-based nerve microelectrode and the advantage that the hardness of the flexible nerve microelectrode is close to the hardness of biological tissue, so that the microelectrode is easy for implantation into biological tissue and not easy to result in immune reaction of biological tissue, and the manufacturing method is simple and is easy to be operated.

Hereinafter, the embodiments of the present disclosure are described in detail with reference to the accompanying drawings. It should be noted that the same reference numerals in different drawings are used to refer to the same elements already described.

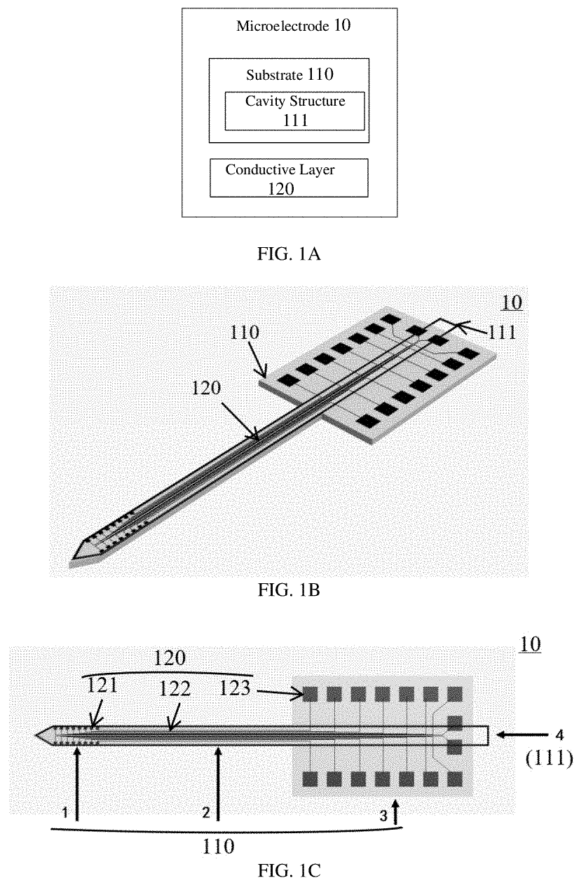

FIG. 1A is a schematic block diagram of the microelectrode provided by at least one embodiment of the present disclosure. For example, as illustrated in FIG. 1A, the microelectrode 10 includes a substrate 110 and a conductive layer 120. The conductive layer 120 is on the substrate 110, and the conductive layer 120 is configured to conduct an electrical signal. The substrate 110 is a flexible substrate and includes a cavity structure 111. The cavity structure 111 is configured to contain or release a fluid. The hardness of the substrate 110 in the case where the cavity structure 111 contains the fluid is different from the hardness of the substrate 110 in the case where the cavity structure 111 does not contain the fluid.

FIG. 1B is a schematic perspective view of the microelectrode provided by at least one embodiment of the present disclosure, and FIG. 1C is a top view of the microelectrode provided by at least one embodiment of the present disclosure.

With reference to FIG. 1B, the microelectrode 10 provided by at least one embodiment of the present disclosure includes the substrate 110 and the conductive layer 120. The conductive layer 120 is on the substrate 110, and is configured to conduct the electrical signal. The material of the conductive layer 120 may be, for example, metal and alloy thereof, or other applicable conductive materials. The substrate 110 has functions such as support, protection, etc. The substrate 110 is the flexible substrate and includes the cavity structure 111, and the cavity structure 111 is configured to contain or release the fluid. The hardness of the substrate 110 in the case where the cavity structure 111 contains the fluid is different from the hardness of the substrate 110 in the case where the cavity structure 111 does not contain the fluid. For example, the hardness of the substrate 110 in the case where the cavity structure 111 contains the fluid is the first hardness, the hardness of the substrate 110 in the case where the cavity structure 111 does not contain the fluid is the second hardness, and the first hardness is larger than the second hardness.

It should be noted that in various embodiments of the present disclosure, the term “hardness” refers to the implant hardness. For the substrate 110, the hardness can be understood as the overall softness presented by the substrate 110. In the present disclosure, the hardness of the substrate 110 is the overall softness presented by the factors such as the elastic modulus of the substrate material and the suffered pressure when filled with fluid. For biological tissue (such as brain tissue), the hardness can be the elastic modulus of the biological tissue itself. For the silicon-based electrode and flexible electrode, generally, the hardness can be the elastic modulus of their substrate materials. In general, the elastic modulus of brain tissue ranges from 1 kPa to 10 kPa, the elastic modulus of the silicon-based electrode (i.e., the silicon-based substrate) is more than 100 GPa, and the elastic modulus of the flexible electrode (i.e., the flexible substrate) ranges approximately from 1 GPa to 10 GPa. Therefore, the elastic modulus of the common flexible electrode is closer to the elastic modulus of brain tissue.

For the microelectrode 10 provided by the embodiments of the present disclosure, the substrate 110 is the flexible substrate. In the case where the fluid is contained in the cavity structure 111, the first pressure suffered by the substrate 110 can reach 100 kPa to 300 kPa. In this case, the hardness of the substrate 110 is the first hardness, which is approximately the sum of the first pressure and the elastic modulus of the flexible substrate itself. Therefore, the hardness of the substrate 110 is largely harder than that of brain tissue, thereby facilitating the implantation of the microelectrode 10 into brain tissue. In the case where there is no fluid in the cavity structure 111, the second pressure suffered by the substrate 110 is 0 kPa. In this case, the hardness of the substrate 110 is the second hardness, which is approximately equal to the elastic modulus of the flexible substrate itself, that is, the softness of the substrate 110 is equivalent to that of the flexible substrate. In this case, the microelectrode 10 is relatively soft and easy to be bent, which is close to the hardness of brain tissue, thereby avoiding causing the immune response of brain tissue.

It should be noted that, in the present disclosure, the hardness of the substrate 110 is positively related to both the elastic modulus of the substrate material itself and the suffered pressure when filled with fluid, and the specific relationship among them is not specifically described in various embodiments of the present disclosure, which can be determined according to actual needs.

Therefore, before the microelectrode 10 provided by the embodiments of the present disclosure is implanted into the biological tissue (such as the brain tissue), the fluid (such as air) can be filled into the cavity structure 111, so that the overall hardness of the microelectrode 10 is improved, and thus the microelectrode 10 can be directly implanted into the biological tissue like the silicon-based nerve microelectrode. After the microelectrode 10 is implanted into the biological tissue, the fluid in the cavity structure 111 can be released, so that the overall hardness of the microelectrode 10 is reduced and the hardness of the flexible microelectrode is restored. The microelectrode 10 after releasing the fluid has good ductility, is easy to deform, adapts to the shape of the tissue structure, and can achieve close fitting, thus avoiding the rejection of the biological tissue and even the inflammation caused by the immune reaction, because of excessive hardness. Therefore, the microelectrode 10 provided by the embodiments of the present disclosure combines the advantage of the mature implantation method of the silicon-based nerve microelectrode and the advantage that the hardness of the flexible nerve microelectrode is close to the hardness of the biological tissue, so that the microelectrode is easy for implantation into the biological tissue and not easy to result in the immune reaction of the biological tissue, and the manufacturing method is simple and is easy to be operated.

With reference to FIG. 1C, in the microelectrode 10 provided by at least one embodiment of the present disclosure, the substrate 110 includes a site region 1, a transition region 2, and a connection region 3. The conductive layer 120 on the substrate 110 includes a site part 121, a conductive part 122, and a connection part 123. For example, as illustrated in FIG. 1C, the site part 121 is located in the site region 1, the conductive part 122 is located in the transition region 2, and the connection part 123 is located in the connection region 3. For example, in some embodiments, the site part 121 is configured to collect and/or output the electrical signal, the connection part 123 is configured to input and/or output the electrical signal, and the conductive part 122 is configured to transmit the electrical signal between the site part 121 and the connection part 123.

For example, in some embodiments, as illustrated in FIG. 1C, the site part 121 includes a plurality of electrode points, the conductive part 122 includes a plurality of connection lines, and the connection part 123 includes a plurality of connection points. The plurality of electrode points, the plurality of connection lines, and the plurality of connection points are in one-to-one correspondence, one end of the connection line is electrically connected to the corresponding electrode point, and the other end of the connection line is electrically connected to the corresponding connection point.

For example, in some embodiments, the plurality of electrode points in the site part 121 may be a group of metal electrode point arrays for the stimulation or recording of the nerve electrical signal, the plurality of connection points in the connection part 123 may be metal welding points, and the plurality of connection lines in the conductive part 122 may be serpentine metal thin wires connecting the electrode points and the welding points. For example, in some embodiments, the electrode points, the connection lines, and the connection points are all formed of electroplated metal materials, and the metal materials may be gold, platinum or platinum-iridium alloy, etc. Of course, the electrode points, the connection lines, and the connection points can also be formed of other conductive materials, such as carbon nanotube (CNT) polymer, conductive polymer, etc. For example, in some embodiments, in order to avoid scratching the surrounding tissue after the microelectrode is implanted, the electrode points can be designed without sharp edges and corners. For example, the plurality of electrode points can be arranged in an array, the plurality of connection points can also be arranged in an array, and part of the plurality of connection lines in the transition region 2 can be arranged in parallel with each other. With regard to the specific materials and structures of the electrode points, the connection lines, and the connection points, the embodiments of the present disclosure are not strictly limited, which can be set according to actual requirements.

For example, in some examples, in the case where it is required to collect the nerve electrical signal of biological tissue, the plurality of electrode points in the site part 121 can be used to collect the nerve electrical signal, and the nerve electrical signal is transmitted to the plurality of connection points in the connection part 123 through the plurality of connection lines in the conductive part 122. The plurality of connection points are electrically connected to the processing circuit provided separately, so that the nerve electrical signal can be transmitted to the processing circuit for subsequent processing and analysis.

For example, in some other examples, in the case where it is required to apply the electrical signals to the biological tissue, the plurality of connection points in the connection part 123 receive the electrical signals provided by the processing circuit provided separately, and the plurality of connection lines in the conductive part 122 transmit the electrical signals to the plurality of electrode points in the site part 121. The plurality of electrode points are in direct contact with the biological tissue, thus these electrical signals can be applied to the biological tissue so as to stimulate the biological tissue to perform corresponding neural activities.

With reference to FIG. 1C, in some embodiments of the present disclosure, the cavity structure 111 included in the microelectrode 10 is located in the region indicated by the number 4 in FIG. 1C, and it can be seen that the cavity structure 111 spans the site region 1, the transition region 2, and the connection region 3 in the microelectrode 10. The cavity structure 111 is configured to contain and release the fluid. For example, the cavity structure 111 is located at the bottom of the substrate 110 (e.g., covered by the substrate 110 in FIG. 1C).

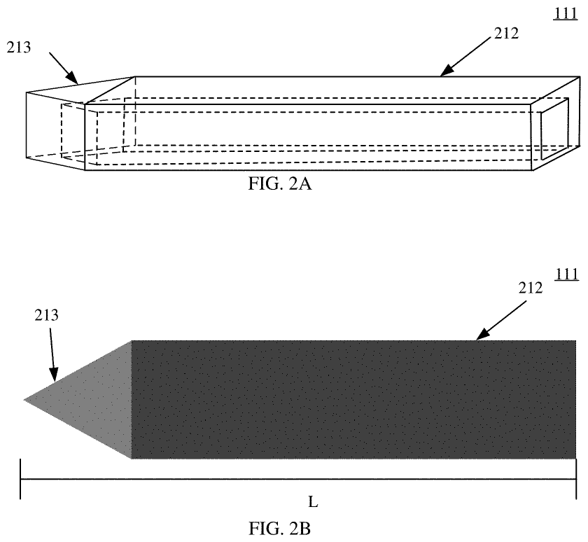

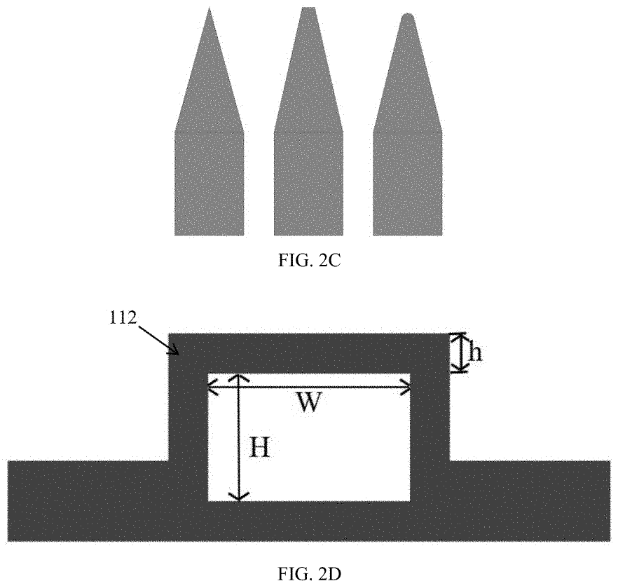

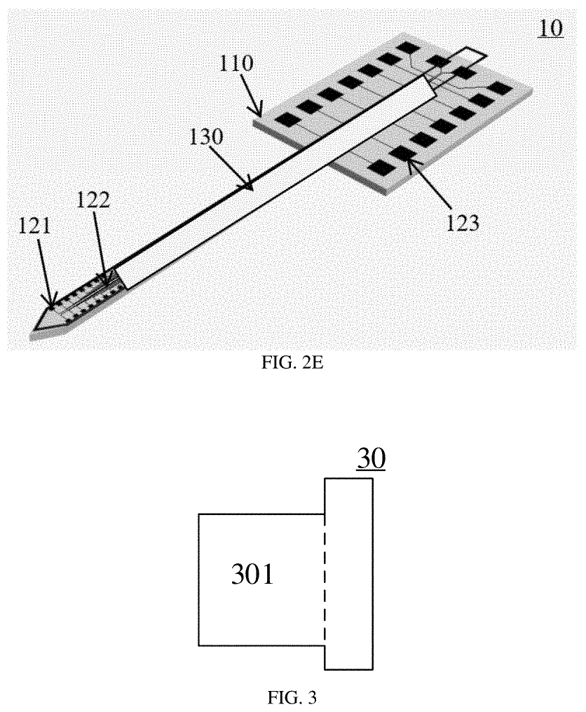

FIG. 2A is a schematic diagram of the cavity structure included in the microelectrode provided by at least one embodiment of the present disclosure, FIG. 2B is a top view of the cavity structure included in the microelectrode provided by at least one embodiment of the present disclosure, FIG. 2C is a top view of other possible forms of cavity structures included in the microelectrode provided by at least one embodiment of the present disclosure, and FIG. 2D is a schematic diagram of an open end of the cavity structure included in the microelectrode provided by at least one embodiment of the present disclosure.

With reference to FIG. 2A and FIG. 1C, for example, in some embodiments of the present disclosure, one end of the cavity structure 111 is an open end, and the other end of the cavity structure 111 is a closed end. For example, the open end is located in the connection region 3 illustrated in FIG. 1C, and the closed end is located in the site region 1 illustrated in FIG. 1C.

For example, in some embodiments of the present disclosure, the cavity structure 111 includes a first cavity 212 and a second cavity 213, and the first cavity 212 and the second cavity 213 are communicated with each other. As illustrated in FIG. 2A and FIG. 2B, the first cavity 212 has a cuboid shape and is located in the transition region 2, the connection region 3, and the site region 1 illustrated in FIG. 1C. While the second cavity 213 is located in the site region 1 illustrated in FIG. 1C, and the second cavity 213 has a tip shape at the closed end.

For example, in the embodiments illustrated in FIG. 2A and FIG. 2B, the shape of the second cavity 213 at the closed end is triangular prism, however, the tip shape is not limited to the triangular prism. For example, in some embodiments, as illustrated in FIG. 2C, the second cavity 213 at the closed end may have an inverted trapezoidal shape or a tapered shape. It should be noted that the triangular prism, inverted trapezoidal shape, and tapered shape illustrated in the embodiments of the present disclosure are only schematic, and are not intended to limit the specific shape of the tip shape. In addition, in some embodiments, the shape of the first cavity 212 may also be a cylinder, which is not limited by the embodiments of the present disclosure.

In the embodiments of the present disclosure, the shape of the second cavity 213 at the closed end is designed as a tip, so that it is easier for the microelectrode 10 to be implanted into the biological tissue from the closed end after the cavity structure 111 is filled with the fluid (that is, after the hardness is improved). In addition, in order to avoid scratching the surrounding biological tissue, the tip-shaped top can be designed as a curved surface. Therefore, the embodiments of the present disclosure do not strictly limit the specific shape of the tip-shaped top, as long as the microelectrode 10 is easy to be implanted into the biological tissue after the hardness of the microelectrode 10 is improved.

With reference to FIG. 2B and FIG. 2D, for example, in some embodiments of the present disclosure, the width W of the first cavity 213 of the microelectrode 10 ranges from 30 microns to 90 microns. For example, in some embodiments of the present disclosure, the height H of the first cavity 213 of the microelectrode 10 ranges from 10 microns to 90 microns. For example, in some embodiments of the present disclosure, the length L of the cavity structure 111 of the microelectrode 10 is equal to the length of the substrate 110. For example, in some embodiments of the present disclosure, the substrate 110 of the microelectrode 10 includes an insulating wall 112, the insulating wall 112 surrounds the cavity structure 111, and the thickness h of the insulating wall 112 ranges from 1 micron to 6 microns.

It should be noted that the specific height H, width W, and length L of the cavity structure 111 and the thickness h of the insulating wall 112 in the microelectrode 10 provided by the embodiments of the present disclosure can be adjusted according to the actual situations, which is not strictly limited by the embodiments of the present disclosure.

For example, in some embodiments, the fluid may be air or a single component gas, such as argon, oxygen, etc. For example, in some embodiments, the fluid may also be a liquid, such as a solution having medicinal properties.

It should be noted that the composition of the fluid described in the present disclosure can be determined according to the actual needs, as long as the overall hardness of the microelectrode 10 is improved after the fluid is filled into the cavity structure 111 and the effect of facilitating implantation is achieved. Therefore, the embodiments of the present disclosure do not specifically limit this.

In the microelectrode 10 provided by at least one embodiment of the present disclosure, the substrate 110 is a flexible substrate. For example, the material of the substrate 110 is a polymer material, such as polyimide, parylene, or photo-sensitive epoxy resin photoresist (such as the SU-8 glue), or the combination of a plurality of polymer materials.

The substrate made of the flexible material allows the microelectrode 10 provided by at least one embodiment of the present disclosure to have good flexibility and ductility after the fluid is released, which is easy to deform, adapts to the shape of the tissue structure, and realizes close fitting. Its good ductility can also ensure that the electrode point moves along with the deformation of tissue, so that the relative position between the electrode point and the target cell is basically fixed, avoiding the dislocation of recording or stimulation because of the tissue deformation, and the hardness is close to the hardness of the biological tissue, so that the microelectrode 10 can coexist with the biological tissue naturally.

For example, in some embodiments of the present disclosure, as illustrated in FIG. 2E, the microelectrode 10 further includes a protective layer 130. The protective layer 130 covers the conductive part 122 and exposes the site part 121 and the connection part 123, and has functions such as protection, shielding, insulation, etc. The material of the protective layer 130 may adopt, for example, a polymer material. For example, the material of the protective layer 130 may be the same as or different from the material of the substrate 110.

It should be noted that, in some embodiments of the present disclosure, the protective layer 130 may cover the entire conductive part 122. For example, the protective layer 130 may cover all connection lines in the conductive part 122. In some other embodiments of the present disclosure, the protective layer 130 may cover part of the conductive part 122, for example, the protective layer 130 may cover part of the connection lines in the conductive part 122, which is not limited by the embodiments of the present disclosure.

FIG. 3 is a schematic diagram of the occluding device for the microelectrode provided by at least one embodiment of the present disclosure.

The occluding device 30 provided by at least one embodiment of the present disclosure is used to cooperate with the microelectrode 10 provided by the embodiments of the present disclosure. For example, the occluding device 30 is configured to close the cavity structure 111 after the cavity structure 111 is filled with the fluid, so that the cavity structure 111 contains the fluid, and to open the cavity structure 111 so that the fluid in the cavity structure 111 flows out.

As illustrated in FIG. 3, for example, in some embodiments, the occluding device 30 may be a piston plate adapted to the open end of the cavity structure 111. For example, in some embodiments, in the case where the occluding device 30 closes the cavity structure 111, the element indicated by reference numeral 301 in FIG. 3 is inserted into the cavity structure 111 so as to tightly close the open end of the cavity structure 111. For example, in some other embodiments, the occluding device 30 may be an airbag piston adapted to the open end of the cavity structure 111.

It should be noted that the embodiments of the present disclosure do not limit the specific structure of the occluding device 30, as long as the occluding device 30 can realize the function of closing the cavity structure 111 after the cavity structure 111 is filled with the fluid, so that the cavity structure 111 can contain the fluid, and opening the cavity structure 111 so that the fluid in the cavity structure 111 can flow out. For example, the occluding device 30 may be made of an elastic material, such as rubber, and the embodiments of the present disclosure do not limit the material of the occluding device.

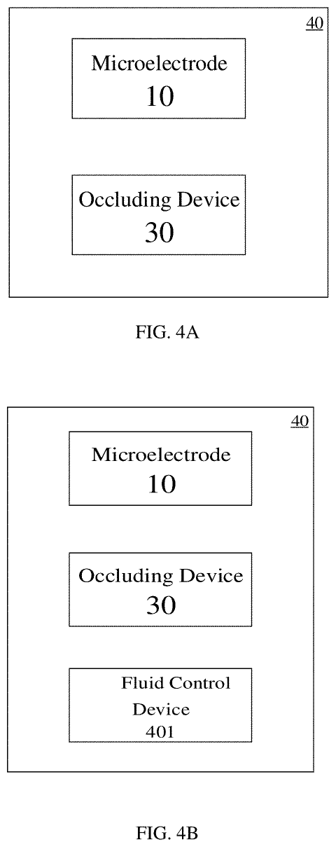

FIG. 4A is a schematic block diagram of the microelectrode system provided by at least one embodiment of the present disclosure, and FIG. 4B is a schematic block diagram of another microelectrode system provided by at least one embodiment of the present disclosure.

As illustrated in FIG. 4A, the microelectrode system 40 provided by at least one embodiment of the present disclosure includes the microelectrode 10 described in any of the above embodiments and the occluding device 30 described in the above embodiments. The microelectrode 10 and the occluding device 30 cooperate with each other. For detailed description, reference can be made to the above contents, which is not repeated here.

As illustrated in FIG. 4B, for example, in some embodiments of the present disclosure, the microelectrode system 40 may include a fluid control device 401 in addition to the microelectrode 10 and the occluding device 30. The fluid control device 401 is configured to inject the fluid into the cavity structure 111 or suck the fluid out of the cavity structure 111. The embodiments of the present disclosure do not limit the specific structure of the fluid control device 401, as long as the fluid control device 401 can realize the function of injecting or sucking the fluid into or out of the cavity structure 111. For example, the fluid control device 401 may be an air pump, a liquid pump, etc.

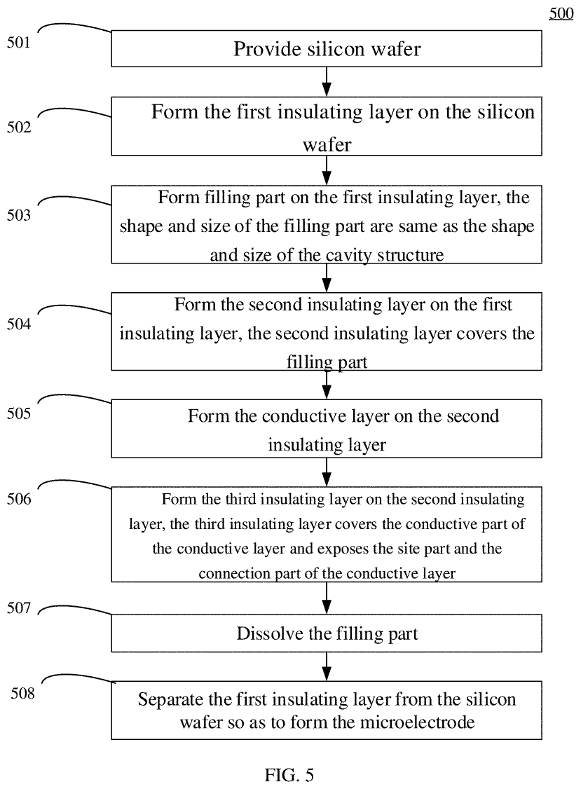

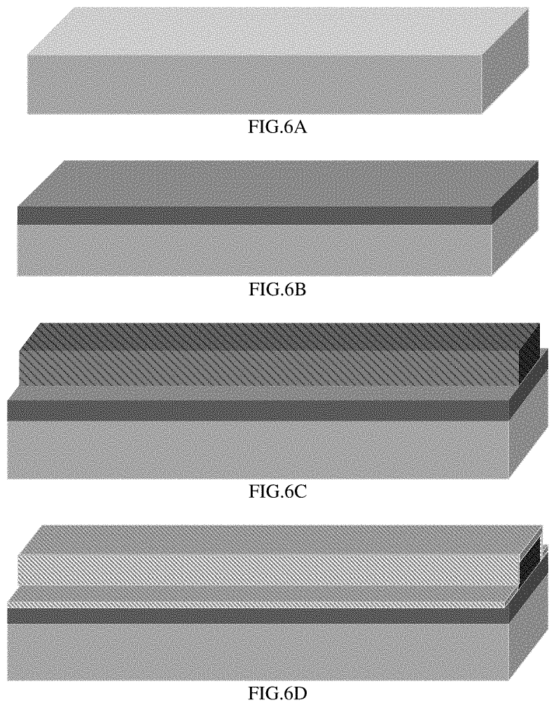

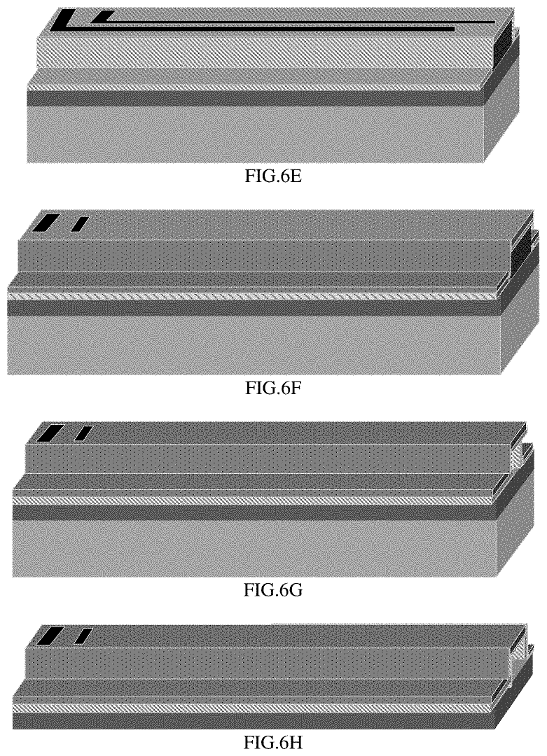

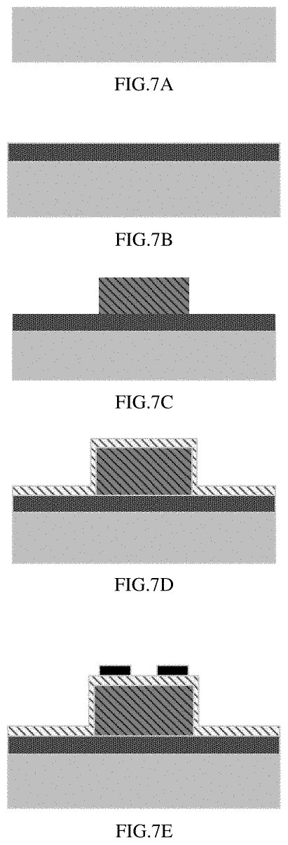

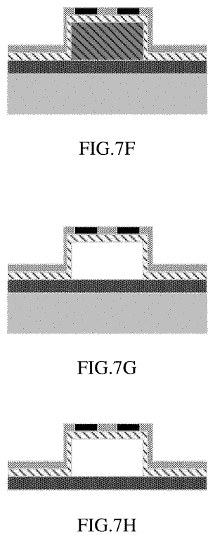

FIG. 5 is a flow chart of a method for manufacturing the microelectrode according to any embodiment provided by at least one embodiment of the present disclosure, FIGS. 6A-6H are perspective schematic views of the microelectrode provided in at least one embodiment of the present disclosure in the manufacturing process, and FIGS. 7A-7H are schematic cross-sectional views of the microelectrode provided in at least one embodiment of the present disclosure in the manufacturing process.

With reference to FIG. 5 and FIGS. 6A-7H, the method 500 for manufacturing the microelectrode 10 provided by at least one embodiment of the present disclosure is described in detail below. The method 500 includes the following operations.

In step 501, a silicon wafer is provided.

As illustrated in FIG. 6A and FIG. 7A, for example, in some embodiments, the standard silicon wafer is used as a supporting substrate.

In step 502, the first insulating layer is formed on the silicon wafer.

As illustrated in FIG. 6B and FIG. 7B, for example, in some embodiments, the first insulating layer is deposited on the silicon wafer. The material of the first insulating layer may be at least one of parylene, polyimide, and photo-sensitive epoxy resin photoresist (such as the SU-8 glue). For example, before deposition, the silicon wafer can also be cleaned and dried, etc.

In step 503, a filling part is formed on the first insulating layer, and the shape and size of the filling part are the same as the shape and size of the cavity structure.

As illustrated in FIG. 6C and FIG. 7C, for example, in some embodiments, the shape of the cavity structure in micro size is defined by the photoresist through the photolithography process, that is, the filling part is formed by the photoresist.

In step 504, the second insulating layer is formed on the first insulating layer, and the second insulating layer covers the filling part.

As illustrated in FIG. 6D and FIG. 7D, for example, in some embodiments, the second insulating layer is deposited on the first insulating layer, and the second insulating layer also covers the filling part on the first insulating layer. For example, the material of the second insulating layer can also be at least one of parylene, polyimide, and photo-sensitive epoxy resin photoresist (such as the SU-8 glue).

In step 505, a conductive layer is formed on the second insulating layer.

As illustrated in FIG. 6E and FIG. 7E, for example, in some embodiments, the micro-electrodes and wires are manufactured on the surface of the second insulating layer by the micro-nano machining processes such as the photolithography, electron beam evaporation, stripping, etc. That is, the site part, the conductive part, and the connection part in the conductive layer are manufactured on the surface of the second insulating layer by using the micro-nano machining processes.

In step 506, the third insulating layer is formed on the second insulating layer, and the third insulating layer covers the conductive part of the conductive layer and exposes the site part and the connection part of the conductive layer.

As illustrated in FIG. 6F and FIG. 7F, for example, in some embodiments, the third insulating layer is deposited on the second insulating layer, and the site part and the connection part of the conductive layer are exposed by using the photolithography process (such as the developing and etching process) so as to facilitating inputting and outputting the electrical signal. For example, the material of the third insulating layer may be at least one of parylene, polyimide, and photo-sensitive epoxy resin photoresist (such as the SU-8 glue).

In step 507, the filling part is dissolved.

As illustrated in FIG. 6G and FIG. 7G, for example, in some embodiments, the material of the filling part is the photoresist. In this case, the applicable solution such as acetone can be used to remove the photoresist, i.e., dissolve the filling part, so as to form the desired cavity structure. It should be noted that the material of the filling part can also be other materials, as long as the material can be dissolved or sacrificed in the subsequent steps to form the desired cavity structure, which is not specifically limited by the embodiments of the present disclosure.

In step 508, the first insulating layer is separated from the silicon wafer so as to form the microelectrode.

As illustrated in FIG. 6H and FIG. 7H, for example, in some embodiments, the salt solution is used to electrolyze the silicon wafer so as to release the whole microelectrode from the silicon wafer.

It should be noted that the substrate described in the above embodiments includes the first insulating layer and the second insulating layer, while the third insulating layer is equivalent to the protective layer described in the above embodiments. In addition, the materials of the first insulating layer, the second insulating layer, and the third insulating layer may be the same polymer material.

It should be noted that the manufacturing process described in the embodiments is only exemplary, and some related steps can be replaced, added or omitted based on the operation described in the embodiments, which is not specifically limited by the embodiments of the present disclosure. It should be noted that the drawings of the embodiments of the present disclosure are only exemplary, and the thickness and size of each material layer in the drawings can be determined according to actual requirements, and the embodiments of the present disclosure do not specifically limit this. It should also be noted that, in the embodiments of the present disclosure, the above steps S501-S508 can be performed sequentially or in other adjusted orders, and some or all of the operations in steps S501-S508 can also be performed in parallel. The embodiments of the present disclosure do not limit the execution order of each step, which can be adjusted according to the actual situations. For example, in some examples, some steps from step S501 to step S508 may be selectively performed, and some additional steps other than step S501 to step S508 may also be performed, which is not specifically limited by the embodiments of the present disclosure.

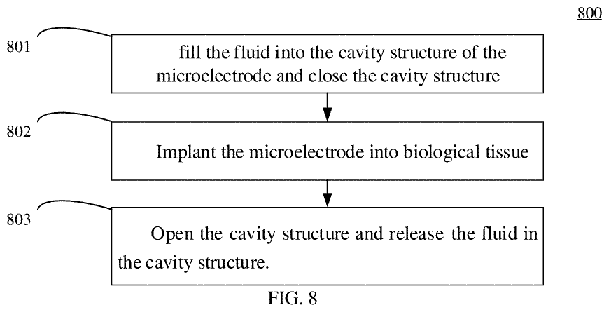

FIG. 8 is a flow chart of the method for using the microelectrode provided by at least one embodiment of the present disclosure.

As illustrated in FIG. 8, the method 800 for using the microelectrode provided by at least one embodiment of the present disclosure includes the following operations.

In step 801, the cavity structure of the microelectrode is filled with the fluid and the cavity structure is closed.

For example, in some embodiments, the fluid (e.g., air, oxygen, or solution, etc.) is filled into the cavity structure of the microelectrode. For example, the operation of filling the fluid into the cavity structure of the microelectrode can be implemented by using the fluid control device described in the above embodiments. For example, the cavity structure is closed by the occluding device described in the above embodiments (for example, the piston plate or airbag piston, etc.), so that the overall hardness of the microelectrode is improved, for example, the hardness of the microelectrode is one order of magnitude higher than the harness of the biological tissue, so that the microelectrode is easy to be implanted into the biological tissue.

In step 802, the microelectrode is implanted into the biological tissue.

For example, in some embodiments, the hardness of the microelectrode after being filled with the fluid is improved, so the mature implantation method for the silicon-based nerve microelectrode can be used to implant the microelectrode after being filled with the fluid into the biological tissue. For example, in some embodiments, the direct insertion implantation of the microelectrode is realized by the precise three-dimensional displacement thruster. It should be noted that the embodiments of the present disclosure do not specifically limit the implantation method.

In step 803, the cavity structure is opened and the fluid in the cavity structure is released.

For example, in some embodiments, the occluding device (e.g., the piston plate or airbag piston, etc.) is separated from the cavity structure, thereby opening the open end of the cavity structure. For example, in some embodiments, the fluid control device described in the above embodiments is used to implement the operation of releasing the fluid from the cavity structure of the microelectrode. For example, the fluid control device can be used to suck the fluid out of the cavity structure. In this case, the hardness of the microelectrode after the fluid is released is approximately equal to the hardness of the general flexible microelectrode, thus the microelectrode after the fluid is released has the advantages of the general flexible microelectrode, such as good flexibility and ductility, and can be closely attached to the biological tissue. Therefore, the microelectrode provided by the embodiments of the present disclosure combines the advantage of the mature implantation method of the silicon-based nerve microelectrode and the advantage that the hardness of the flexible nerve microelectrode is close to the hardness of the biological tissue, so that the microelectrode is easy for implantation into the biological tissue and not easy to result in the immune reaction of the biological tissue, and the method for manufacturing or using the microelectrode is simple and is easy to be operated.

It should be noted that, in the embodiments of the present disclosure, the above steps S801-S803 can be performed in sequence or in other adjusted sequences, and some or all of the operations in steps S801-S803 can also be performed in parallel. The embodiments of the present disclosure do not limit the perform sequence of each step, which can be adjusted according to the actual situations. For example, in some examples, some steps from step S801 to step S803 may be selectively performed, and some additional steps other than step S801 to step S803 may also be performed, which is not specifically limited by the embodiments of the present disclosure.

For the present disclosure, the following statements should be noted.

(1) The accompanying drawings involve only the structure(s) in connection with the embodiment(s) of the present disclosure, and other structure(s) can be referred to common design(s).

(2) In case of no conflict, the embodiments of the present disclosure and features in the embodiments may be combined with each other to obtain new embodiments.

What are described above are related to the specific implementations of the present disclosure only and not limitative to the scope of the disclosure, and the scope of the disclosure is defined by the accompanying claims.

1. A microelectrode, comprising:
a substrate; and
a conductive layer on the substrate, configured to conduct an electrical signal,
wherein the substrate is a flexible substrate and comprises a cavity structure, the cavity structure is configured to contain or release a fluid, and hardness of the substrate in a case where the cavity structure contains the fluid is different from hardness of the substrate in a case where the cavity structure does not contain the fluid.

2. The microelectrode according to claim 1, wherein the substrate comprises a site region, a transition region, and a connection region;
the conductive layer comprises a site part, a conductive part, and a connection part, the site part is configured to collect and/or output the electrical signal, the connection part is configured to input and/or output the electrical signal, and the conductive part is configured to transmit the electrical signal between the site part and the connection part; and
the site part is in the site region, the conductive part is in the transition region, and the connection part is in the connection region.

3. The microelectrode according to claim 2, wherein the cavity structure is in the site region, the transition region, and the connection region.

4. The microelectrode according to claim 2, wherein one end of the cavity structure is an open end, other end of the cavity structure is a closed end, the open end is in the connection region, and the closed end is in the site region.

5. The microelectrode according to claim 4, wherein the cavity structure comprises a first cavity and a second cavity, and the first cavity and the second cavity are communicated with each other;
the first cavity has a cuboid shape, and is in the transition region, the connection region, and the site region; and
the second cavity is in the site region, and the second cavity has a tip shape at the closed end.

6. The microelectrode according to claim 5, wherein the tip shape comprises a triangular prism shape, a tapered shape, or an inverted trapezoidal shape.

7. The microelectrode according to claim 5, wherein a width of the first cavity ranges from 30 microns to 90 microns.

8. The microelectrode according to claim 5, wherein a height of the first cavity ranges from 10 microns to 90 microns.

9. The microelectrode according to claim 1, wherein a length of the cavity structure is equal to a length of the substrate.

10. The microelectrode according to claim 1, wherein the fluid comprises air, single component gas, or liquid.

11. The microelectrode according to claim 1, wherein a material of the substrate comprises a polymer, and the polymer comprises polyimide, parylene, or photo-sensitive epoxy resin photoresist.

12. The microelectrode according to claim 1, wherein the substrate comprises an insulating wall, the insulating wall surrounds the cavity structure, and a thickness of the insulating wall ranges from 1 micron to 6 microns.

13. The microelectrode according to claim 2, wherein the site part comprises a plurality of electrode points, the conductive part comprises a plurality of connection lines, and the connection part comprises a plurality of connection points,
the plurality of electrode points, the plurality of connection lines, and the plurality of connection points are in one-to-one correspondence, one end of a connection line is electrically connected to a corresponding electrode point, and other end of the connection line is electrically connected to a corresponding connection point.

14. The microelectrode according to claim 2, further comprising a protective layer,
wherein the protective layer covers the conductive part and exposes the site part and the connection part.

15. An occluding device for the microelectrode according to claim 1,
wherein the occluding device is configured to close the cavity structure after the cavity structure is filled with the fluid so that the cavity structure contains the fluid, and to open the cavity structure so that the fluid in the cavity structure flows out.

16. A microelectrode system, comprising the microelectrode according to claim 1 and an occluding device for the microelectrode,
wherein the occluding device is configured to close the cavity structure after the cavity structure is filled with the fluid so that the cavity structure contains the fluid, and to open the cavity structure so that the fluid in the cavity structure flows out.

17. The microelectrode system according to claim 16, further comprising a fluid control device,
wherein the fluid control device is configured to inject the fluid into the cavity structure or suck the fluid out of the cavity structure.

18. A method for manufacturing the microelectrode according to claim 1, comprising:
providing a silicon wafer;
forming a first insulating layer on the silicon wafer;
forming a filling part on the first insulating layer, wherein a shape and size of the filling part are same as a shape and size of the cavity structure;
forming a second insulating layer on the first insulating layer, wherein the second insulating layer covers the filling part;
forming the conductive layer on the second insulating layer;
forming a third insulating layer on the second insulating layer, wherein the third insulating layer covers a conductive part of the conductive layer and exposes a site part and a connection part of the conductive layer;
dissolving the filling part; and
separating the first insulating layer from the silicon wafer so as to form the microelectrode,
wherein the substrate comprises the first insulating layer and the second insulating layer.

19. The method according to claim 18, wherein materials of the first insulating layer, the second insulating layer, and the third insulating layer are a same polymer material, and a material of the filling part is photoresist.

20. (canceled)

21. A method for using the microelectrode according to claim 1, comprising:
filling the fluid into the cavity structure of the microelectrode and closing the cavity structure;
implanting the microelectrode into biological tissue; and
opening the cavity structure and releasing the fluid in the cavity structure.
